(19) United States
(12) Patent Application Publication
(10) Pub. No.: US 2013/0267484 A1
(43) Pub. Date: Oct. 10, 2013

(54) TREATMENT OF RHEUMATOID ARTHRITIS WITH MASITINIB

(71) Applicants: Alain Moussy, Paris (FR); Jean-Pierre Kinet, Aix-en-provence (FR)

(72) Inventors: Alain Moussy, Paris (FR); Jean-Pierre Kinet, Aix-en-provence (FR)

(73) Assignee: AB SCIENCE, PARIS (FR)

(21) Appl. No.: 13/700,788

(22) PCT Filed: Jun. 1, 2011
(86) PCT No.: PCT/EP2011/059034

§ 371 Date: Jun. 27, 2013

Related U.S. Application Data
(60) Provisional application No. 61/350,603, filed on Jun. 2, 2010.

(51) Int. Cl. A61K 31/496; A61K 45/06; A61K 31/519

(52) U.S. Cl. CPC A61K 31/496; A61K 31/519; A61K 45/06
USPC 514/171; 514/253.1; 514/249

(57) ABSTRACT

The present invention relates to a tyrosine kinase inhibitor or a mast cell inhibitor, and in particular masitinib or a pharmaceutically acceptable salt thereof, for the treatment of human rheumatoid arthritis.

The present invention relates to the treatment of rheumatoid arthritis (RA). The present invention relates to the administration of at least one tyrosine kinase inhibitor or mast cell inhibitor, and in particular of masitinib or a pharmaceutically acceptable salt thereof, in an appropriate dosage regimen for the treatment of RA.

### BACKGROUND OF THE INVENTION

Rheumatoid Arthritis

Inflammatory arthritic diseases, in particular RA, are an important health problem. RA is an autoimmune disorder characterized by a chronic and persistent inflammation that can result in progressive joint destruction, deformity, disability and premature death. The pivotal clinical manifestation of RA is a polyarticular synovitis, which is a consequence of the underlying cellular and molecular inflammatory events leading to pain, swelling due to synovial thickening and effusion, and stiffness of the joints. RA has a profoundly negative impact on the patient's quality of life, as well as significant economic and societal implications. Estimates of adult RA prevalence across Europe range from 0.1 to 1.2%, whilst a 2008 survey of prevalence in the United States revealed that RA affects 0.6% (1.3 million persons) of U.S. adults. Moreover, since RA is an age related condition, its prevalence is projected to increase in line with demographic trends.

Assessment of Rheumatoid Arthritis

The American College of Rheumatology (ACR) has defined the following criteria for the classification of rheumatoid arthritis (Arnett et al. 1988). At least four of the seven criteria have to be met for classification as RA. Criteria 1 through 4 must have been present for at least 6 weeks.
Morning stiffness: Morning stiffness in and around the joints, lasting at least 1 hour before maximal improvement.
Arthritis of 3 or more joint areas: At least 3 joint areas simultaneously have had soft tissue swelling or fluid (not bony overgrowth alone) observed by a physician; the 14 possible joint areas are right or left proximal interphalangeal (PIP) joints, metacarpophalangeal (MCP) joints, wrist, elbow, knee, ankle, and metatarsophalangeal (MPT) joints.
Arthritis of hand joints: At least 1 area swollen (as defined above) in a wrist, MCP or PIP joint.
Symmetric arthritis: Simultaneous involvement of the same joint areas (see 2 above) on both sides of the body (bilateral involvement of PIPs, MCPs, or MTPs is acceptable without absolute symmetry).
Rheumatoid nodules: Subcutaneous nodules, over bony prominences, or extensor surfaces, or in juxta-articular regions, observed by a physician.
Serum rheumatoid factor: Demonstration of abnormal amounts of serum rheumatoid factor by any method for which the result has been positive in <5% of normal control subjects.
Radiographic changes: Radiographic changes typical of RA on posteroanterior hand and wrist radiographs, which must include erosions or unequivocal bony decalcification localized to or most marked adjacent to the involved joints (osteoarthritis changes alone do not qualify).

In 1991 the ARC published revised and validated criteria for the classification of global functional status in RA (Hochberg et al. 1992). These criteria were originally developed as an adjunct to criteria for staging of RA, for the purpose of classifying patients at entry into therapeutic trials. The objective of these revised criteria was to provide a quick and simple classification of functional capacity during the clinical evaluation of patients with RA and possibly for the determination of work disability.

Class I: Completely able to perform usual activities of daily living (self-care, vocational, and avocational)*. *Self-care activities include dressing, feeding, bathing, grooming, and toileting. Avocational (recreational and/or leisure) and vocational (work, school, homemaking) activities are patient-desired and age- and sex-specific.

Class II: Able to perform usual self-care and vocational activities, but limited in avocational activities.

Class III: Able to perform usual self-care activities, but limited in vocational and avocational activities.

Class IV: Limited ability to perform usual self-care, vocational, and avocational activities.

It has been shown that joint damage is a consequence of disease activity over time. Categories or states of high, moderate, and low disease activity as well as remission have been identified for the most commonly used indices. Indeed, the lower the disease activity category that can be attained under therapy, the lower the progression of joint damage. There is no single ‘gold standard’ quantitative measure to assess and monitor the clinical status or disease activity in patients with RA. Therefore, a variety of measures have been used in clinical research and clinical care, including laboratory tests, radiographic scores, formal joint counts, physical measures of functional status, global measures and patient self-report questionnaires. These measures may address disease activity, joint damage, both activity and damage, or long-term outcomes. Two quantitative indices that are widely used in clinical trials are the (i) ACR Core Data Set (Felson et al. 1993), which includes swollen joint count, tender joint count, physician assessment of global status, acute-phase reactant—erythrocyte sedimentation rate or C-reactive protein, functional status, pain, patient estimate of global status, a radiograph in studies over 1 year or longer, and (ii) the disease activity score (DAS) (van der Heijde et al., 1993; Prevoo et al., 1995), which includes a swollen joint count, tender joint count, acute-phase reactant, and patient assessment of global status. For example, higher DAS28 values are indicative of greater disease activity with significance placed on the threshold values of: DAS28<2.6; 2.6≦DAS28≦3.2; 3.2<DAS28≦5.1; and DAS28>5.1, corresponding to the classifications of remission, inactive RA, moderate RA and very active RA, respectively (Prevoo et al., 1995).

One major limitation of existing criteria is that they are primarily intended to categorize established RA patients for clinical research and where not developed for use in clinical practice. The main concern is that they do not perform well in the context of early inflammatory arthritis. Moreover, it is now well established that early initiation of sometimes aggressive therapy can prevent erosions and may occasionally induce remission, even if this means treating people who don't fulfill the ACR criteria. For example, a patient may suffer from persistent, inflammatory arthritis but does not meet the current classification criteria even though their disease is persistent and functionally disabling. Recently a joint ACR and European League Against Rheumatism (EULAR) task force announced a new criteria for RA appropriate for newly presenting patients with inflammatory polyarthritis. These new diagnostic criteria are expected to replace the current criteria and help standardize the diagnosis of RA, bringing it into line with the prevailing clinical practices.

An outline of the new diagnostic criteria was described at the American College of Rheumatology 2009 Annual Meeting as follows (ACR Clinical Symposia. Program and abstracts of the American College of Rheumatology 2009 Annual Meeting; Oct. 17-21, 2009; Philadelphia, Pa.). The new criteria, rate patients on a scale of 0-10 points, with points assigned in four separate domains of signs and symptoms: joint involvement, serology, duration of symptoms, and acute phase reactants. Patients are diagnosed as having ‘definite’ RA if they score 6 or more points according to the following criteria:

Joint Involvement:

1 medium-large joint (0 points); 2-10 medium-large joints (1 point); 1-3 small joints (2 points); 4-10 small joints (3 points); more than 10 small joints (5 points).

Serology:

Not positive for either rheumatoid factor or anti-citrullinated protein antibody (0 points); At least one of these two tests are positive at low titer, defined as more than the upper limit of normal but not higher than three times the upper limit of normal (2 points); at least one test is positive at high titer, defined as more than three times the upper limit of normal (3 points).

Duration of Synovitis:

Less than 6 weeks (0 points); 6 weeks or longer (1 point).

Acute Phase Reactants:

Neither C-reactive protein nor erythrocyte sedimentation rate is abnormal (0 points); abnormal CRP or abnormal ESR (1 point)

Note: Patients receive the highest point level they fulfill within each domain. For example, a patient with five small joints involved and four large joints involved scores 3 points. A score of 3 or 4 points will most likely be adopted to distinguish patients with ‘probable RA’ from those in whom RA is ‘improbable’.

Treatment of Rheumatoid Arthritis

RA has a complex etiopathogenesis necessitating that a patient's treatment be individually and continually tailored for effective management. There are three general classes of drugs commonly used in the treatment of RA: non-steroidal anti-inflammatory agents (NSAIDs), corticosteroids, and disease modifying anti-rheumatic drugs (DMARDs). To date, the etiology of RA remains unresolved and there are no known cures. Rather, treatment attempts to alleviate symptoms, e.g. NSAIDs and corticosteroids, or to slow disease progression by modifying the disease process, e.g. DMARDs. The treatment of RA still utilizes NSAIDs and analgesics to relieve pain and stiffness, NSAIDs reduce acute inflammation, thereby decreasing pain and improving function, and have independent analgesic properties. However, it has been recognized that these agents have limited effects on slowing the progression of destructive RA. In contrast, DMARDs improve symptoms and retard erosive damage. In cases of clearly defined RA, clinicians now initiate these agents as soon as possible and may use them in combination to provide maximum benefit. DMARDs favorably influence the clinical evolution of the disease, enhance biological signs and for some, even slow down the radiological progression of lesions; their mechanism of action generally involving suppression of the body's overactive immune and/or inflammatory systems. DMARDs have become the cornerstone of RA treatment, with the therapeutic goals of preventing or controlling joint damage, prevent loss of function and decrease pain.

Since the 90s, methotrexate (MTX) has been considered the reference treatment of RA. However, it has failed to achieve adequate disease control whether used alone or in combination with other DMARDs. In many RA subjects a lack of efficacy and intolerability to currently available DMARDs has been reported, indicating a need for improved therapy. One shortcoming of MTX is that since it is not a remission-inducing drug, disease progression continues unabated with the consequence that after an initial respite, the patient returns to their pre-treatment condition over the course of months to a few years. A problem more general to DMARDs is that of drug resistance, which represents a major obstacle to the effective long-term management of RA. Typically, when conventional DMARDs fail to yield sufficient efficacy then a potent subclass of DMARDs, known as biologics, are recommended. Such therapies generally act by selectively inhibiting, depleting or blocking at least one critical mediator of RA pathogenesis, e.g. tumor necrosis factor alpha (TNFα) inhibition via Adalimumab; IL-1 blocking via Anakinra; tyrosine kinase inhibition via Imatinib; B-cell depletion via Rubuximab; or T-cell activation blocking via Abatacept; to name but a few. In circumstances where these also fail, due to primary (inherent) or secondary (acquired) resistance, combination DMARD strategies sometimes bring about synergistic benefits.

Despite these approved therapies, the unmet medical need in the RA field remains substantial. Several reasons can be given for this:
None of the available drugs cure or completely stop the disease process and in the best circumstances are not particularly effective in controlling the disease.
The most recently approved treatments for RA such as anti TNFα are primarily injectable solutions which, considering the chronic nature of this disease, impact negatively on the patient's adherence to treatment, quality-of-life and can lead to a common side effect of injection site reactions.
RA follows a highly heterogeneous disease progression, yet patient-optimized treatment, e.g. weight-adjusted dosing, is not developed in the currently available drugs.
Long-term treatment regimens using corticosteroids or DMARDs are associated with numerous detrimental side effects, with its benefits possibly outweighed by potential complications. Important adverse events in clinical trials are reported for existing treatments, including injection site reactions, infections (respiratory), headache, nausea, sinusitis, rhinitis, rash, abdominal pain, asthenia, diarrhea, hepatotoxicity, and cytopenia.
Both conventional and biologic DMARDs, including MTX and anti-TNFα, may become inefficient for controlling disease activity in severe RA.

Thus, beyond the already developed therapeutic strategies, there exists an imperative need to identify alternative RA treatments that demonstrate high efficacy over time in monotherapy, exploit novel therapeutic targets for more effective combination therapies, minimize toxicity and are affordable. One such approach involves blocking intracellular proinflammatory messages, which is currently represented by the strategy of selective protein tyrosine kinase (TK) inhibition.

Role of c-Kit and Mast Cells in Inflammation

Many cell populations participate in the pathogenesis of inflammatory arthritic diseases; however, recently there is a growing body of evidence implicating mast cells (MC) as being one of the major contributors. MCs are predominantly found in tissues at the interface between the host and the external environment, such as lung, connective tissue, lymphoid tissue, gut mucosa, and skin. They develop from a common circulating CD34+/c-Kit+/CD13+/FcεRI− hematopoietic progenitor representing a single lineage, which gives rise to different phenotypes after migrating into peripheral tissues. Immature MC progenitors circulate in the bloodstream and differentiate in tissues. These differentiation and proliferation processes are influenced by cytokines, notably Stem Cell Factor (SCF), also termed Kit ligand (KL), Steel factor (SL) or Mast Cell Growth Factor (MCGF). The SCF receptor is encoded by the proto-oncogene c-Kit. It has been shown that SCF regulates the migration, maturation, proliferation, and activation of MCs in vivo—injection of recombinant SCF into rodents, primates, or humans, results in an increase in MC numbers at both the site of injection and at distant sites.

Binding of SCF to the c-Kit receptor induces c-Kit dimerization followed by its transphosphorylation, leading to the recruitment and activation of various intracytoplasmic substrates. These activated substrates induce multiple intracellular signaling pathways responsible for cell proliferation and activation. MCs are characterized by their heterogeneity, not only regarding tissue location and structure but also at functional and histochemical levels. MC activation is followed by the controlled release of a variety of mediators that are essential for the defense of the organism against invading pathogens. By contrast, in the case of hyperactivation of MCs, uncontrolled hypersecretion of these mediators is deleterious for the body. MCs produce a large variety of mediators categorized here into three groups:
Preformed granule-associated mediators (histamines, proteoglycans, and neutral proteases);
Lipid-derived mediators (prostaglandins, thromboxanes and leucotrienes);
Various cytokines (including the interleukins: IL-1, IL-2, IL-3, IL-4, IL-5, IL-6, IL-8 and tumor necrosis factor alpha TNF-α, GM-CSF, MIP-1α, MIP-1β and IFN-γ).

Human MCs constitutively express a number of receptors for different biological molecules. Among these receptors, whose ligation induces the activation of MCs, the best known is the high affinity receptor for IgE (FcεRI). Binding of IgE-multivalent antigen complexes to FcεRI leads to receptor aggregation and internalization, signaling, and degranulation. This can be accompanied by the transcription of cytokine genes, thus, perpetuating the inflammatory response. Moreover, triggering of MCs leads to the secretion of diverse pre-formed and/or de novo synthesized mediators, such as vasoactive amines (histamine, serotonin), sulfated proteoglycans, lipid mediators (prostaglandin D2, leucotrienes), growth factors, proteases, cytokines and chemokines as described previously. These mediators can, alone or in synergy with macrophage-derived and T cell-derived cytokines, generate a complex inflammatory response and induce the recruitment and activation of inflammatory cells to the site of degranulation.

### AIMS OF THE INVENTION

The invention aims to solve the technical problem of providing an active ingredient for the treatment of RA, and in particular an effective treatment for DMARD-refractory active RA.
The invention also relates to the treatment of such a disease in a human patient.
The invention aims to provide an efficient treatment for such a disease at an appropriate dose, route of administration and daily intake.
The invention also aims to solve the technical problem of providing an active ingredient that improves prior art methods for the treatment of RA.

### SUMMARY OF THE INVENTION

The invention relates to a tyrosine kinase inhibitor or a MC inhibitor, in particular masitinib or a pharmaceutically acceptable salt thereof, for the treatment of RA in human patients, including DMARD-refractory active RA, wherein masitinib is to be administered daily at a starting dose of 3.0 to 7.5 mg/kg/day (but may be decreased or increased by up to 1.5 mg/kg/day, i.e. 3.0 to 7.5±1.5 mg/kg/day), or a starting dose of 3.0 to 6.5±1.5 mg/kg/day, or even a starting dose of 3.0 to 6.0±1.5 mg/kg/day.

The invention also relates to a method of treatment of RA in human patients, including DMARD-refractory active RA, wherein a tyrosine kinase inhibitor or a MC inhibitor, and in particular masitinib or a pharmaceutically acceptable salt thereof, is to be administered daily at a starting dose of 3.0 to 7.5±1.5 mg/kg/day, or of 3.0 to 6.5±1.5 mg/kg/day, or even of 3.0 to 6.0±1.5 mg/kg/day.

In another embodiment, the invention also relates to a method of treatment of RA in human patients, including DMARD-refractory active RA, wherein a tyrosine kinase inhibitor or a MC inhibitor, and in particular masitinib or a pharmaceutically acceptable salt thereof, is administered for the treatment of RA in combination with at least one other antirheumatic; for example, NSAIDs, corticosteroids and DMARDs, including MTX or anti-TNFα.

### DESCRIPTION OF THE INVENTION

Mast Cells in Rheumatoid Arthritis

There is a growing body of evidence implicating MCs as major contributors to the pathogenesis of RA. MCs may be considered the immunological sentinel of the synovium, acting immediately in the event of joint trauma by liberating an array of proinflammatory mediators. However, MCs also appear to perpetuate the chronic process by their marked increased accumulation in the synovial lining of the inflamed joint and their ability to produce numerous proinflammatory cytokines and growth and angiogenic factors. Some of the most compelling evidence for the connection of MCs to RA comes from studies in the K/B×N murine model, an animal model of autoantibody-induced arthritis, which has demonstrated that MC-deficient mice are resistant to arthritis, with susceptibility restored following MC engraftment (Lee et al., 2002). This model has also been used to show how MCs contribute to the initiation of joint inflammation by elaboration of interleukin-1 (IL1) (Nigrovic et al., 2007). Thus, MCs may function as a cellular link between autoantibodies, soluble mediators, and other effector populations in inflammatory arthritis. The mechanism by which MC might play a role in the induction of RA could be their extraordinary ability to release inflammatory cytokines, and in particular TNF-α, or degrading enzymes that have been shown to be involved in the inflammatory course of the disease. For instance, antibodies against TNF-α have revealed interesting anti-RA activity in vivo. As such, MCs represent an attractive therapeutic target.

Masitinib is a Potent Mast Cell Inhibitor

Masitinib is a small molecule selectively inhibiting specific tyrosine kinases such as c-kit, PDGFR, Lyn and to a lesser extent the fibroblast growth factor receptor 3 (FGFR3), without inhibiting, at therapeutic doses, kinases associated with known toxicities (i.e. those tyrosine kinases or tyrosine kinase receptors attributed to possible tyrosine kinase inhibitor cardiac toxicity, including ABL, KDR and Src) (Dubreuil et al, 2009). The chemical name for masitinib is 4-(4-methylpiperazin-1-ylmethyl)-N-[4-methyl-3-(4-pyridin-3ylthiazol-2-ylamino) phenyl]benzamide—CAS number 790299-79-5, and the structure is shown below.

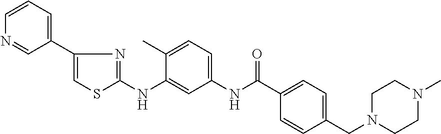 [SMILES: Cc1ccc(NC(=O)c2ccc(CN3CCN(C)CC3)cc2)cc1Nc1nc(-c2cccnc2)cs1]

Masitinib was first described in U.S. Pat. No. 7,423,055 and EP1525200B1. A detailed procedure for the synthesis of masitinib mesilate is given in WO2008/098949.

Masitinib's strong inhibitory effect on wild-type and juxtamembrane-mutated c-Kit receptors, results in cell cycle arrest and apoptosis of cell lines dependent on c-Kit signaling (Dubreuil et al., 2009). Stem cell factor, the ligand of the c-Kit receptor, is a critical growth factor for MCs, essential to their survival, proliferation, differentiation, adhesion and degranulation processes (Reber et al., 2006). Thus, masitinib is an effective antimastocyte, exerting a direct antiproliferative and pro-apoptotic action on MCs through its inhibition of c-Kit signaling. In addition to its antiproliferative properties, masitinib can also regulate the activation of MCs through its targeting of Lyn and Fyn, key components of the transduction pathway leading to IgE induced degranulation (Gilfillan & Tkaczyk, 2006). This can be observed in the inhibition of FcεRI-mediated degranulation of human cord blood MCs (Dubreuil et al., 2009).

Treatment of Rheumatoid Arthritis with at Least One Tyrosine Kinase Inhibitor or a Mast Cell Inhibitor, in Particular Masitinib or a Pharmaceutically Acceptable Salt Thereof.

MCs play a prominent role in all the inflammatory processes and actively participate in the pathogenesis of RA, in part because they release large amounts of various mediators that sustain the inflammatory network. SCF, the ligand of the c-KIT receptor, is a critical growth factor for MCs; hence, there exists a strong relation between the SCF/MC c-KIT pathway and the pathogenesis of RA. Thus, molecules able to inhibit the survival and/or activation of MCs may be able to control the symptoms and progression of RA or any related disease. In connection with the present invention, we consider that masitinib is fulfilling this role in the treatment of RA via, but not limited to, inflammatory-mediated mechanisms, through its inhibition of both c-Kit and Lyn kinase activity and by consequence, inhibition of MC proliferation and activation. This could limit the role of MCs in RA and reduce the inflammation linked to MCs degranulation. The mechanism of action of masitinib is original and there is currently no other drug directed against these targets in RA in phase 3 clinical trials.

A “patient” according to the invention is in particular a human.

Masitinib is particularly active against RA and RA symptoms.

In connection with the present invention, it would seem, without wishing to be bound by the theory, that a tyrosine kinase inhibitor or a MC inhibitor, in particular masitinib or a pharmaceutically acceptable salt thereof, also provides protection against RA in DMARD-refractory patients via its inhibition of inflammatory-mediated and immune-mediated mechanisms.

The present invention relates to the use of a tyrosine kinase inhibitor or a MC inhibitor, in particular masitinib or a pharmaceutically acceptable salt thereof, for the treatment of RA in human patients, wherein masitinib is to be administered daily at a starting dose of 3.0 to 7.5±1.5 mg/kg/day, or of 3.0 to 6.5±1.5 mg/kg/day, or even of 3.0 to 6.0±1.5 mg/kg/day, and preferably wherein said patients are diagnosed as having ‘definite’ or ‘probable’ RA according to the ACR/EULAR classification systems and an ACR global functional status of class I to III.

One embodiment of the invention is a combined administration of at least one tyrosine kinase inhibitor or a MC inhibitor, in particular masitinib or a pharmaceutically acceptable salt thereof, with at least one antirheumatic drug, i.e. NSAIDs, corticosteroids and/or DMARDs.

The invention also relates to a method of treatment of RA in human patients, wherein a tyrosine kinase inhibitor or a MC inhibitor, and in particular masitinib or a pharmaceutically acceptable salt thereof, is to be administered daily at a starting dose of 3.0 to 7.5±1.5 mg/kg/day, or of 3.0 to 6.5±1.5 mg/kg/day, or even of 3.0 to 6.0±1.5 mg/kg/day, and preferably wherein said patients are diagnosed as having definite or probable RA according to the ACR/EULAR classification systems and an ACR global functional status of class I to III.

In one embodiment said tyrosine kinase inhibitor or MC inhibitor, and in particular masitinib or a pharmaceutically acceptable salt thereof, is administered for the treatment of RA, and in particular for the treatment of DMARD-refractory active RA, including patients resistant to MTX and/or anti-TNFα.

In another embodiment said tyrosine kinase inhibitor or MC inhibitor, and in particular masitinib or a pharmaceutically acceptable salt thereof, is administered for the treatment of RA in combination with at least one antirheumatic drug at an appropriate dose, including MTX.

Advantageously, in the use or the method above, said patients have an ACR global functional status of class I to III. Patients according to the invention are those afflicted with ‘definitive RA’ or ‘probable RA’ according to the ACR and/or EULAR classification systems; more specifically according to the new ACR/EULAR classification system (ACR Clinical Symposia. Program and abstracts of the American College of Rheumatology 2009 Annual Meeting; Oct. 17-21, 2009; Philadelphia, Pa.) with scores of between 3 to 10; or 4 to 10; or even 6 to 10.

In one preferred embodiment, masitinib is masitinib mesilate. Regarding best dosage regimen, said tyrosine kinase inhibitor or MC inhibitor, and in particular masitinib or a pharmaceutically acceptable salt thereof, is to be administered at a starting daily dose of 3.0 to 7.5±1.5 mg/kg/day; or of 3.0 to 6.5±1.5 mg/kg/day, or even of 3.0 to 6.0±1.5 mg/kg/day nonetheless said tyrosine kinase inhibitor or MC inhibitor, and in particular masitinib or a pharmaceutically acceptable salt thereof, can be dose escalated by increments of 1.5 mg/kg/day to reach a maximum of 9.0 mg/kg/day in low responder patients

Particularly, the method of the invention is for patients diagnosed as having definite or probable RA according to the ACR/EULAR classification systems and an ACR global functional status of class I to III

Indeed, depending on age, individual condition, mode of administration, and the clinical setting, effective doses of said tyrosine kinase inhibitor or MC inhibitor, and in particular masitinib or a pharmaceutically acceptable salt thereof, in human patients with RA are 3.0 to 9.0 mg/kg/day per os, preferably in two daily intakes. For adult human patients with active DMARD-refractory RA, a starting dose of said tyrosine kinase inhibitor or MC inhibitor, and in particular masitinib or a pharmaceutically acceptable salt thereof, of 4.5 to 6.0 mg/kg/day has been found to be the preferred embodiment according to the invention. For patients with an inadequate response after an assessment of response to therapy and in the absence of limiting toxicities, dose escalation of said tyrosine kinase inhibitor or MC inhibitor, and in particular masitinib or a pharmaceutically acceptable salt thereof to a maximum of 9.0 mg/kg/day can be safely considered and patients may be treated as long as they benefit from treatment and in the absence of limiting toxicities.

Dose adjustment can be considered a dynamic process, with a patient undergoing multiple increases and/or decreases to optimize the balance between response and toxicity throughout treatment, both of which are likely to vary over time and duration of drug exposure. If dose escalation is undertaken, it is suggested that the starting dose of 3.0 to 6.0±1.5 mg/kg/day be incremented by 1 to 2 mg/kg/day up to a maximum dose of 9.0 mg/kg/day, over a period which depends upon clinical observations. For example, a single dose escalation of said tyrosine kinase inhibitor or MC inhibitor and in particular masitinib or a pharmaceutically acceptable salt thereof, and preferably masitinib mesilate may take from 1 to 2 months. It is also contemplated herein that to fully obtain the therapeutic benefits of a patient-optimized dose of said tyrosine kinase inhibitor or MC inhibitor, and in particular masitinib or a pharmaceutically acceptable salt thereof, dose increments smaller than 1 to 2 mg/kg/day could be implemented. Dose reduction is to be considered to reduce toxicity in appropriate cases.

Any dose indicated herein refers to the amount of active ingredient as such, not to its salt form.

Pharmaceutically acceptable salts are pharmaceutically acceptable acid addition salts, like for example with inorganic acids, such as hydrochloric acid, sulfuric acid or a phosphoric acid, or with suitable organic carboxylic or sulfonic acids, for example aliphatic mono- or di-carboxylic acids, such as trifluoroacetic acid, acetic acid, propionic acid, glycolic acid, succinic acid, maleic acid, fumaric acid, hydroxymaleic acid, malic acid, tartaric acid, citric acid or oxalic acid, or amino acids such as arginine or lysine, aromatic carboxylic acids, such as benzoic acid, 2-phenoxy-benzoic acid, 2-acetoxy-benzoic acid, salicylic acid, 4-aminosalicylic acid, aromatic-aliphatic carboxylic acids, such as mandelic acid or cinnamic acid, heteroaromatic carboxylic acids, such as nicotinic acid or isonicotinic acid, aliphatic sulfonic acids, such as methane-, ethane- or 2-hydroxyethane-sulfonic, in particular methanesulfonic acid (or mesilate), or aromatic sulfonic acids, for example benzene-, p-toluene- or naphthalene-2-sulfonic acid.

In a preferred embodiment of the above-depicted treatment, the active ingredient masitinib is administered in the form of masitinib mesilate; which is the orally bioavailable mesylate salt of masitinib—CAS 1048007-93-7 (MsOH); C28H30N6OS.CH3SO3H; MW 594.76:

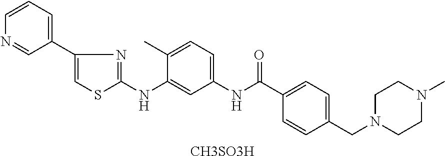

Given that the masitinib dose in mg/kg/day used in the described dose regimens refers to the amount of active ingredient masitinib, compositional variations of a pharmaceutically acceptable salt of masitinib mesilate will not change the said dose regimens.

Masitinib may be administered via different routes of administration but oral administration is preferred. Thus, in still another preferred embodiment, in the use or the method above, masitinib or salts thereof, is administered orally; preferably twice a day for long term period such as over more than 3 months, preferably more than 12 months. Masitinib can be administered in the form of 100 and 200 mg tablets.

In one embodiment the invention relates to a method as defined in the present description, including examples and claims, wherein said tyrosine kinase inhibitor or a MC inhibitor, in particular masitinib or a pharmaceutically acceptable salt thereof, is administered in combination with at least one antirheumatic drug; including, but not limited to, NSAIDs, corticosteroids and DMARDs,

“Combination” refers to a combined administration with the same galenic formulation and also a separate administration, i.e. in separate galenic formulation.

Disease-modifying antirheumatic drugs include conventional (or non-biological) DMARDs and biologic DMARDs. Conventional DMARDs include MTX, sulfasalazine, hydroxychloroquine, leflunomide, cyclosporine, azathioprine and minocycline.

Methotrexate (MTX):

Due to its long-term effectiveness and demonstrated ability to slow disease progression, MTX is the most commonly prescribed DMARD. MTX acts within weeks to months and its clinical effects are dose-dependent, which allows for dose titration to control disease activity. Typically, clinicians start the medication at a dose of 7.5 to 15 mg/week. Then one escalates the dose to a maximum tolerated dose of 20 to 25 mg. One may improve both tolerability and bioavailability with parenteral (subcutaneous/intramuscular) administration. Patients should also receive folate supplementation (1 to 5 mg/day) to reduce the toxicity of the drug. The most common side effects include oral ulcers, nausea, diarrhea, altered liver function tests, alopecia and idiosyncratic pulmonary reactions. According to the ACR guidelines, clinicians should monitor patients every 1 to 2 months with lab work, including CBC, liver function tests and serum creatinine.

Leflunomide:

Leflunomide is used at a dose of 10 to 20 mg/day. Side effects mainly include nausea and diarrhea. Toxicities include altered liver function tests, cytopenias and teratogenicity.

Hydroxychloroquine:

Hydroxychloroquine is commonly dosed at 200 mg twice a day (maximum 6.5 mg/kg). This is generally considered the safest DMARD although it also has the least evidence to support radiographic benefit. No lab monitoring is needed. Side effects are minimal and toxicities mainly include retinal toxicity. Accordingly, these patients should have annual eye exams.

Sulfasalazine:

Generally prescribe as two or three divided doses daily up to 2 to 3 g/day. Side effects include nausea and diarrhea. One can limit these effects by emphasizing a slow escalation of dosing over several weeks. Toxicities include suppression of the bone marrow and liver toxicity requiring routine laboratory monitoring.

Combination DMARDs

have been shown to be more effective than monotherapy in various trials. “Triple therapy” (MTX, sulfasalazine and hydroxychroloquine), and “the COBRA regimen” (sulfasalazine, MTX and high-dose oral steroids) have both proven to be better than monotherapy. Biologic DMARDs include infliximab (Remicade, Centocor), etanercept (Enbrel, Amgen/Wyeth), adalimumab (Humira, Abbott), abatacept (Orenica, Bristol-Myers Squibb) and rituximab (Rituxan, Genentech), while many more are in development. Currently available TNF-α inhibitors include infliximab, adalimumab and etanercept. Infliximab and adalimumab are both monoclonal anti-TNF antibodies. Etanercept is a fusion protein consisting of two p75 TNF receptors coupled to the Fc portion of a standard human IgG1 immunoglobulin molecule. As the name TNF inhibitor implies, all of these agents bind to TNF molecules and prevent interaction with its receptors on target cell surfaces, thus limiting propagation of the autoimmune inflammatory response. Clinicians dose etanercept at 50 mg sc weekly, adalimumab at 40 mg sc once every 1 to 2 weeks, and administer infliximab via infusion at 3-10 mg/kg every 4 to 8 weeks. Common minor adverse events include injection site reactions with etanercept and adalimumab, and infusion reactions with infliximab. Rare adverse events include optic neuritis, multiple sclerosis-like sequelae, aplastic anemia, interstitial lung disease, lupus like syndrome and hepatotoxicity. Overall, patients taking TNF inhibitors are at an increased risk for infections, particularly of the skin and respiratory tract. The risk of cancers associated with anti-TNF agents is controversial. Researchers have reported solid malignancies and an increased risk of lymphomas with all TNF inhibitors. However, there is also a pre-existing association of malignancies in patients with active RA. Currently, it is considered wise to avoid these agents in patients who have a history of malignancy. Newer DMARD developments include the costimulation inhibitor abatacept and the anti-CD20 antibody rituximab. Abatacept modulates the immune response by binding to CD80/86 on antigen presenting cells (such as dendritic cells, macrophages or B cells). Accordingly, this agent prevents costimulatory binding of CD28 and prevents full T cell activation.

The currently available DMARDs include: Methotrexate (Rheumatrex®, Trexall®); Hydroxychloroquine (Plaquenil®); Sulfasalazine (Azulfidine®); Leflunomide (Arava®); Tumor Necrosis Factor Inhibitors—etanercept (Enbrel®, adalimumab (Humira®), and infliximab (Remicade®); T-cell Costimulatory Blocking Agents—abatacept (Orencia®); B cell Depleting Agents—rituximab (Rituxan®); Interleukin-1 (IL-1) Receptor Antagonist Therapy—anakinra (Kineret®); and Intramuscular Gold. Other Immunomodulatory and cytotoxic agents include azathioprine (Imuran®), cyclophosphamide, and cyclosporine A(Neoral®, Sandimmune®).

In the present invention as defined above, the preferred antirheumatic drug, dosed ideally in accordance to the manufacture's recommendations, are for example, and without particular limitation, either: a NSAID; a non-biological DMARD (e.g. methotrexate [Rheumatrex®, Trexall®]; cyclosporine [Sandimmune®, Neoral®]); a biological DMARD such as TNFα blockers (e.g. etanercept [Enbrel®], infliximab [Remicade®], adalimumab [Humira®]); or a corticosteroid (e.g. prednisone). In this regard, masitinib and at least one antirheumatic drug are to be administered separately, simultaneously or sequentially in time.

This combination of tyrosine kinase inhibitor or a MC inhibitor, in particular masitinib or a pharmaceutically acceptable salt thereof, with at least one DMARD, NSAID, and/or corticosteroid, and especially those mentioned above, is done in a single galenic formulation or alternatively separate, galenic formulation.

NSAIDs reduce acute inflammation, thereby decreasing pain and improving function, and have independent analgesic properties. Examples include: COX-2 inhibitors (e.g. celecoxib, Celebrex®; etoricoxib; Arcoxia®; lumiracoxib, Prexige®), as well as, ibuprofen (Advil®, Motrin®, Nuprin®); naproxen (Alleve®); meloxicam (Mobic®); etodolac (Lodine®); nabumetone (Relafen®); sulindac (Clinoril®); tolementin (Tolectin®); choline magnesium salicylate (Trilasate®); diclofenac (Cataflam®, Voltaren®, Arthrotec®); Diflusinal (Dolobid®); indomethicin (Indocin®); Ketoprofen (Orudis®, Oruvail®); Oxaprozin (Daypro®); and piroxicam (Feldene®). However, NSAIDs alone do not change the course of the disease of RA or prevent joint destruction. Corticosteroids (such as prednisone; methylprenisolone, Medrol®; triamcinolone) have both anti-inflammatory and immunoregulatory activity. However, extended corticosteroid therapy is associated with undesirable side effects and complications upon withdrawal and is therefore often considered as a last resort pharmacological treatment. Both NSAIDs and corticosteroids have a short onset of action while DMARDs can take several weeks or months to demonstrate a clinical effect. Thus, corticosteroids or NSAIDs are useful in early disease as temporary adjunctive therapy while in combination with at least one tyrosine kinase inhibitor or a MC inhibitor, in particular masitinib or a pharmaceutically acceptable salt thereof.

According to a particular embodiment, the composition of the invention is an oral composition.

As is known to the person skilled in the art, various forms of excipients can be used adapted to the mode of administration and some of them can promote the effectiveness of the active molecule, e.g. by promoting a release profile rendering this active molecule overall more effective for the treatment desired.

The pharmaceutical compositions of the invention are thus able to be administered in various forms, more specially for example in an injectable, pulverizable or ingestible form, for example via the intramuscular, intravenous, subcutaneous, intradermal, oral, topical, rectal, vaginal, ophthalmic, nasal, transdermal or parenteral route. A preferred route is oral administration. The present invention notably covers the use of a compound according to the present invention for the manufacture of pharmaceutical composition.

Such medicament can take the form of a pharmaceutical composition adapted for oral administration, which can be formulated using pharmaceutically acceptable carriers well known in the art in suitable dosages. Such carriers enable the pharmaceutical compositions to be formulated as tablets, pills, dragees, capsules, liquids, gels, syrups, slurries, suspensions, and the like, for ingestion by the patient. In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

The present invention is illustrated by means of the following examples

### Example 1

Masitinib in the Treatment of DMARD-Refractory Active Rheumatoid Arthritis

A multicenter, uncontrolled, open-label, randomized, dose ranging, phase 2a trial to evaluate the safety and efficacy of masitinib in the treatment of DMARD-refractory active RA (Tebib et al., 2009).

Methods

Study Design and Treatment

This was a multicentre, prospective, uncontrolled, open-label, randomized, dose ranging, phase 2a study of masitinib in adults with active RA, followed over a 12-week period. Masitinib, supplied as 100 and 200 mg tablets (AB Science, France), was administered orally in two daily intakes. To evaluate the dose response of masitinib in DMARD-refractory active RA, dose ranging was performed by randomly assigning patients into two initial treatment groups of 3 and 6 mg/kg/day (1:1 ratio). Dosage could be increased by 1.5 mg/kg/day at weeks 4 and 8 in cases of insufficient response accompanied by minimal toxicity. Likewise, the dose could be reduced by 1.5 mg/kg/day or treatment discontinued in the case of serious adverse event (SAE). Patients exhibiting a significant improvement after 12 weeks of treatment were eligible to continue receiving treatment after entering a compassionate program, wherein assessments were performed every 4 weeks for the first 3 months of extension, and every 12 weeks thereafter.

Permitted medications for the treatment of possible cutaneous rash and face oedema during the study were hydroxyzine (Atarax) and prednisolone. Other permitted concomitant medications were: one NSAID (including COX-2 inhibitors) at constant dosage; oral corticosteroids at stable doses mg/day; analgesics without anti-inflammatory action or oral narcotic analgesics; and medically acceptable forms of birth control. Physical therapy, if performed at the time of study entry, was provided under a stable and consistent regimen. The following treatments of active RA were prohibited during the study: surgery; DMARD treatment including MTX, anti-TNFα biology therapies, leflunomide, IL1-Ra, azathioprine, and cyclosporine; immunosuppressive drugs; cytotoxic drugs; intramuscular or intravenous steroid injections; corticosteroids intra-articular or soft tissue injection; and alternate investigational drugs or investigational combinations of approved drugs. Drugs that interact with the same CYP450 isoenzymes (2C9, 2D6 and 3A4) as masitinib were prohibited (e.g. acetaminophen) due to the inherent risk of either reduced activity or enhanced toxicity of any concomitant medication. Finally, use of analgesics was prohibited on assessment days, until after all clinical efficacy evaluations had been completed.

Patients

Human patients aged from 18 to 75 years who had been diagnosed with active RA, according to the ACR criteria, for whom disease on-set had occurred after 16 years of age and who had a history of DMARD failure, (predominantly MTX and/or anti-TNFα), or primary resistance to anti-TNFα, were eligible to participate. Their active RA had an ACR functional class of 1-3 and duration of at least 6 months. In addition, patients exhibited ≧8/66 swollen joints, ≧10/68 painful joints, and at least one of the following three conditions: erythrocyte sedimentation rate ≧28 mm/h; C-reactive protein (CRP)≧15 mg/litre; or morning stiffness ≧45 min at both screening and baseline time points. The main exclusion criteria were patients with: inadequate bone marrow function, defined as an absolute neutrophil count ≦2.5×10⁹/litre, and platelets ≦100×10⁹/litre; active current infection, history of infection requiring hospitalisation, history of recurrent infections, or treatment with antibiotics within 2 weeks of screening. Treatment wash-out periods observed prior to entry to the study were: a) DMARD use within 4 weeks; b) five half-lives, or wash-out in accordance for a specific drug, whichever is longer; c) any live (attenuated) vaccines taken within 4 weeks; d) use of more than one NSAID or its dosage change within 4 weeks; e) prednisone or equivalent corticosteroid dosage >10 mg/day or any dosage change within 4 weeks; and f) prednisone or equivalent corticosteroid dosage >20 mg administered via intra-articular injection, bolus intramuscular or intravenous treatment within 4 weeks. Other exclusion criteria included any previous use of recombinant interleukin-1 receptor antagonist (IL1-Ra) and patients who were pregnant or nursing.

Safety and Efficacy Assessment

Safety was assessed by occurrence of adverse events (AE), SAEs, and monitoring biochemical, haematological and urinalysis parameters during the study period, with toxicity graded according to the Common Toxicity Criteria version 3.0. In the event of a SAE (i.e. grade 3 or 4), treatment was interrupted until resolution and then resumed, with a permitted dose reduction of 1.5 mg/kg/day or treatment discontinuation if toxicity recurred. Evaluation of treatment efficacy was based upon the evolution of clinical symptoms associated with active RA at week 12 relative to baseline. Primary endpoints were the ACR response criteria of ACR20, ACR50 and ACR70. For each patient all efficacy parameters were recorded on the first day of treatment (baseline), prior to administration of masitinib, and then again after 4, 8 and 12 weeks of treatment. Secondary endpoints included the 12-week analysis of disease activity score using 28 joint counts (DAS28), index of improvement in RA (ACRn) and CRP improvement. Higher DAS28 values are indicative of greater disease activity with significance placed on the threshold values of: DAS28<2.6≦; DAS28≦3.2; 3.2<DAS28≦5.1; and DAS28>5.1, corresponding to the classifications of remission, inactive RA, moderate RA and very active RA, respectively. CRP is an acute phase reactant and a sensitive serum marker of inflammation. Discrimination between dose regimens was investigated by analysis of the time (days) to first ACR variable response according to initial dosage. Since dose adjustment was permitted at weeks 4 and 8 in cases of insufficient treatment response, the dose at time of first response was also analysed.

Statistical Methods

Efficacy data are presented using descriptive statistics, contrasting initial dosage groups or according to previous DMARD failure. For comparison of groups according to initial dosage on a continuous variable, Student test (with Satterwhaite correction for unequal variance) or Wilcoxon test were used when normality was not rejected or rejected, respectively (normality determined via the Shapiro-Wilk test). For the same comparison on a qualitative variable, Chi-square or Fisher Exact test (if the Chi-square hypotheses were not filled) were used. The rate of patients achieving the various ACR response variables after 12 weeks of treatment (remission rate) are presented in terms of percentage and number of patients. Patients were assigned to either 3 or 6 mg/kg/day treatment groups based upon a randomisation schedule generated for packaging and labelling by the Biostatistics Section of AB Science. Individual treatment doses to be administered were supplied in sealed envelopes to be opened by the investigator at time of inclusion. Patients received the treatment from the investigator on an open basis.

Due to the relatively high patient drop-out rate of this study, analysis was conducted on two different datasets: one with an imputation of missing values according to the last observation carried forward (LOCF) methodology and the other in the absence of data imputation, i.e. the observed cases (OC). Analysis for efficacy was performed on a modified Intention-To-Treat (ITT) population and Per Protocol (PP) population. The ITT population was defined as those patients who had received at least one dose of masitinib and who had undergone at least one post-baseline assessment of efficacy. The PP population was defined as a subgroup of the ITT population that in addition had presented no major protocol deviations and had completed at least 28 days of treatment exposure.

Results

Baseline Characteristics and Participant Flow

Between December 2004 and March 2006, a total of 43 patients were enrolled into the study. Participants were randomly assigned into two initial treatment groups receiving a masitinib dosage of either 3 mg/kg/day (n=22) or 6 mg/kg/day (n=21). Of these, 27/43 patients (63%) completed the study with 21/43 patients (49%) entering the study's extension phase (of which, 10/43 patients (23%) have received treatment for over one year; 8/43 (19%) for over two years; and 3/43 (7%) for over three years). Of the 16 patients (37%) who withdrew before completion of the 12-week study period, occurrence of AE was cited as the primary cause of discontinuation. Participant baseline characteristics, disposition and dosing history are presented in Table 1, according to the randomized dose ranging treatment groups. Baseline values of several efficacy parameters were higher in the 6 mg/kg/day group compared to the 3 mg/kg/day group, e.g. DAS28 was respectively 7.1 against 6.1 (p=0.010); CRP was 62 against 26 (p=0.029); swollen joints was 22.1 against 15.3 (p=0.046); previous anti-TNF was 67% against 36% (p=0.056); and HAQ was 2.2 against 1.9 (p=0.082). Hence, the 6 mg/kg/day initial dosage arm had a higher baseline of disease severity.

Three patients were excluded from the randomized population due to the lack of efficacy data following baseline, thus, in accordance to our ITT population definition, the resulting ITT population was n=40. This corresponded to 3 and 6 mg/kg/day randomized dose ranging groups of n=22 and n=18, respectively. An additional four patients were excluded from the PP population (n=36 with n=18 for each group): one due to a major protocol violation, i.e. treated with prednisone at 20 mg/day before baseline; and three due to insufficient exposure time, i.e. <28 days.

Regarding analysis of the primary efficacy outcome, i.e. ACR score at week 12, 39/40 (97%) patients had sufficient post-baseline data available for analysis in the ITT LOCF group; (the size of this efficacy analysis group differs in size from the ITT population since although the missing patient fulfilled the ITT criteria they did not possess a sufficiently complete dataset to permit calculation of the multiparametric ACR score). The PP OC efficacy analysis group had sufficient data available for analysis of 27/36 (75%) patients. Secondary efficacy outcomes were likewise analyzed in accordance to the number of patients possessing sufficient data for evaluation at 12 weeks.

TABLE 1
Baseline characteristics, overall disposition and dosing history, according to initial dosage*

| | | | |
|---|---|---|---|
| | Masitinib 3 mg/kg/day | Masitinib 6 mg/kg/day | Total population |
| Parameter | (n = 22) | (n = 18) | (n = 40) |

Demographic (Intent-To-Treat population)

| | | | | |
|---|---|---|---|---|
| Age (years) | Mean ± SD | 54.0 ± 12.2 | 55.5 ± 9.2 | 54.7 ± 10.8 |
| | Min-Max | 27.0-75.0 | 34.0-69.0 | 27.0-75.0 |
| Weight (kg) | Mean ± SD | 67.1 ± 12.8 | 69.2 ± 20.5 | 68.1 ± 16.5 |
| | Min-Max | 49.0-88.0 | 50.0-136.0 | 49.0-136.0 |
| Sex | Female | 19/22 (86.4%) | 12/18 (66.7%) | 31/40 (77.5%) |

Clinical (Intent-To-Treat population)

| | | | | |
|---|---|---|---|---|
| Disease duration (years) | Mean ± SD | 11.8 ± 5.9 | 10.7 ± 8.1 | 11.3 ± 6.9 |
| Tender joints | Mean ± SD | 24.7 ± 11.1 | 32.2 ± 16.3 | 28.1 ± 14.0 |
| Swollen joints | Mean ± SD | 15.3 ± 10.4 | 22.1 ± 12.0 | 18.4 ± 11.5 |
| Patient pain assessment | Mean ± SD | 67.4 ± 19.2 | 68.6 ± 27.4 | 67.9 ± 23.0 |
| Patient assessment of DA | Mean ± SD | 69.4 ± 24.9 | 73.0 ± 22.9 | 71.0 ± 23.8 |
| Physicians assessment of DA | Mean ± SD | 66.4 ± 19.5 | 66.8 ± 18.8 | 66.6 ± 18.9 |
| HAQ | Mean ± SD | 1.9 ± 0.6 | 2.2 ± 0.5 | 2.0 ± 0.6 |
| CRP | Mean ± SD | 26.2 ± 28.4 | 62.3 ± 57.6 | 42.3 ± 46.9 |
| DAS28 | Mean ± SD | 6.1 ± 0.8 | 7.1 ± 1.1 | 6.5 ± 1.0 |
| DMARD failures (%) | Anti-TNFα | 8/22 (36.4%) | 12/18 (66.7%) | 20/40 (50.0%) |
| | Other | 14/22 (63.6%) | 6/18 (33.3%) | 20/40 (50.0%) |

Patient Disposition (Randomized population)

| | Masitinib 3 mg/kg/day | | Masitinib 6 mg/kg/day | | Total population | |
|---|---|---|---|---|---|---|
| | (n = 22) | | (n = 21) | | (n = 43) | |
| Early study discontinuation | 7/22 | (31.8%) | 9/21 | (42.9%) | 16/43 | (37.2%) |
| Insufficient therapeutic effect | 1/7 | (14.3%) | 1/9 | (11.1%) | 2/16 | (12.5%) |
| Protocol violation | 0/7 | (0.0%) | 0/9 | (0.0%) | 0/16 | (0.0%) |
| Adverse event | 6/7 | (85.7%) | 7/9 | (77.8%) | 13/16 | (81.3%) |
| Consent withdrawn | 0/7 | (0.0%) | 1/9 | (11.1%) | 1/16 | (6.3%) |
| End of study without extension | 5/22 | (22.7%) | 1/21 | (4.8%) | 6/43 | (14.0%) |
| Entered extension phase | 10/22 | (45.4%) | 11/21 | (52.3%) | 21/43 | (48.9%) |

Dosing Adjustment (Intent-To-Treat population over 12-week study phase)

| | Masitinib 3 mg/kg/day | Masitinib 6 mg/kg/day | Total population |
|---|---|---|---|
| Parameter | (n = 22) | (n = 18) | (n = 40) |
| No dose adjustment | 10/22 (45%) | 8/18 (44%) | 18/40 (45%) |
| Increase by 1.5 mg/kg/day | 6/22 (27%) | 3/18 (17%) | 9/40 (23%) |
| Increase by 3.0 mg/kg/day | 2/22 (9%) | 5/18 (28%) | 7/40 (18%) |
| Increase by 4.5 mg/kg/day | 3/22 (14%) | 0/18 (0%) | 3/40 (8%) |
| ^{†}Other | 1/22 (5%) | 2/18 (11%) | 3/40 (8%) |

*Active rheumatoid arthritis patients were randomised to receive masitinib therapy at initial dosing levels of 3.0 mg/kg/day or 6.0 mg/kg/day, administered per os for 12 weeks. Dose adjustment was permitted depending upon efficacy and safety assessments. Pain and disease activity were assessed using an EQ-5D visual analogue scale.
DA = disease activity;
HAQ = Health Assessment Questionnaire;
CRP = C-reactive protein;
DAS28 = Disease Activity Score in 28 joints;
DMARDs = disease-modifying antirheumatic drugs.
^{†}Other = combination of dose augmentation and/or diminution.

Subgroup analysis of the ITT population with respect to previous DMARD treatment failure revealed that 20/40 patients (50%) were unresponsive to anti-TNFα (including: 5/40 patients (12%) resistant to one anti-TNFα; 10/40 patients (25%) resistant to more than one anti-TNFα; and 5/40 patients (12%) intolerant to anti-TNFα). In addition, 33/40 patients (82%) were unresponsive to MTX. Among them, 18 patients were unresponsive to both anti-TNFα and MTX. Analyses of the participant baseline characteristics with respect to previous treatment failure (data not shown), suggest that although the entire population was classified as having “very active RA”, those patients previously treated with anti-TNFα were suffering from RA of even greater severity than the other patients.

Safety and Tolerability of Masitinib

Assessment of safety was performed on all patients who had received at least one dose of masitinib (n=43) over the study duration, including treatment extension period with a cut-off date of 31 Aug. 2008. Overall patient exposure to masitinib was on average 288±378 days, with a median exposure of 91 days and a range of 8 to 1274 days. The incidence of common (>4%) treatment related AEs according to intensity is presented in Table 2 for the initial (12-week study period) and extension phases. A total of 40/43 patients (93%) reported at least one masitinib related (or not assessable) AE during the initial phase. In general, AEs were transient in nature and of mild to moderate intensity; nevertheless, occurrence of AEs was the main reason for 13/43 patients (30%) discontinuing treatment. In 9/43 patients (21%) the AEs were severe, including edema and rash in 3/43 (7%) and 2/43 patients (5%), respectively. One patient presented with angioedema of moderate intensity (face edema, rash and dyspnea without hypotension or any sign of shock). This event resolved upon masitinib interruption and without specific medications, ruling out any anaphylactic or anaphylactic-like reaction. No changes considered to be of clinical relevance were observed regarding physical, hematological or urinalysis parameters during the initial phase, however, 1/43 (2%) patient presented with hepatic disorder of increased liver enzymes (ASAT: 122, ALAT: 188, and alkaline phosphatase: 635) at a dose of 6 mg/kg/day. This episode, reported as a severe transaminases increase AE, occurred after 14 days treatment and resolved within 4 weeks of drug withdrawal, with no reoccurrence following reintroduction of treatment. Analysis of AEs with respect to the dose of their occurrence (data not shown) showed no clear dose-toxicity relationships exist with the exception of edema. The number of patients experiencing at least one edema was 11/43 (26%), with 6/36 (16.7%) for doses ≦6.0 mg/kg/day and 5/15 (33.3%) for doses >6.0 mg/kg/day. Such edematous episodes typically occurred 4 weeks (median onset time 28 days) after the first drug intake or dose increase and abated within an average of 16 days. Four patients (9%) reported non-fatal SAE of severe intensity suspected to be related to masitinib (or not assessable) consisting of skin rash, pleural effusion, pneumonia and RA flare up. Only one of those SAE (pleural effusion) resulted in patient withdrawal. All these patients recovered without sequelae and no deaths occurred during this study.

For patients entering the extension phase (n=21) a clear decrease in the occurrence of AEs was evident as well as a reduction in severity. Overall, 10/21 patients (48%) reported at least one masitinib related (or not assessable) AE, which were of mild, moderate or severe intensity in 4/21 (19%), 3/21 (14%), and 3/21 patients (14%), respectively. Specifically, no incidence of skin rash, nausea, vomiting or diarrhea was reported after week 12 and occurrence of edema decreased >60%.

TABLE 2
Number of subjects with at least one suspected (or not
assessable) adverse event (>4%), according to intensity
Initial Phase

| System Organ Class/Preferred | | | | |
|---|---|---|---|---|
| Term^{†} | All (n = 43) | Mild | Moderate | Severe |
| At least one suspected AE* | 40 (93.0%) | 29 (67.4%) | 27 (62.8%) | 9 (20.9%) |
| Rash - All | 13 (30.2%) | 7 (16.3%) | 8 (18.6%) | 2 (4.7%) |
| Edema - All | 11 (25.6%) | 2 (4.7%) | 6 (14.0%) | 3 (7.0%) |
| Nausea | 10 (23.3%) | 6 (14.0%) | 5 (11.6%) | |
| Diarrhea | 8 (18.6%) | 5 (11.6%) | 2 (4.7%) | 1 (2.3%) |
| Headache | 6 (14.0%) | 4 (9.3%) | 2 (4.7%) | |
| Abdominal Pain Upper | 5 (11.6%) | 4 (9.3%) | 1 (2.3%) | |
| Vomiting | 5 (11.6%) | 1 (2.3%) | 4 (9.3%) | |
| Asthenia | 5 (11.6%) | | 4 (9.3%) | 1 (2.3%) |
| Pyrexia | 3 (7.0%) | 1 (2.3%) | 1 (2.3%) | 1 (2.3%) |
| Herpes Simplex | 3 (7.0%) | 2 (4.7%) | 1 (2.3%) | |
| Weight Decreased | 3 (7.0%) | 2 (4.7%) | 1 (2.3%) | |
| Dyspnoea | 3 (7.0%) | 1 (2.3%) | 1 (2.3%) | 1 (2.3%) |
| Abdominal Pain | 2 (4.7%) | 1 (2.3%) | | 1 (2.3%) |
| Dry Mouth | 2 (4.7%) | 1 (2.3%) | | 1 (2.3%) |
| Hyperthermia | 2 (4.7%) | | 1 (2.3%) | 1 (2.3%) |
| Gastroenteritis | 2 (4.7%) | | 2 (4.7%) | |
| Blood Creatinine Increased | 2 (4.7%) | 1 (2.3%) | 1 (2.3%) | |
| Cough | 2 (4.7%) | 1 (2.3%) | 1 (2.3%) | |
| Alopecia | 2 (4.7%) | 2 (4.7%) | | |
| Petechiae | 2 (4.7%) | 1 (2.3%) | 1 (2.3%) | |

Extension phase

| System Organ Class/Preferred | | | | |
|---|---|---|---|---|
| Term | All (n = 21) | Mild | Moderate | Severe |
| At least one suspected AE | 10 (47.6%) | 4 (19.0%) | 3 (14.3%) | 3 (14.3%) |
| Edema - All | 2 (9.5%) | | 2 (9.5%) | |
| Leukopenia | 1 (4.8%) | | 1 (4.8%) | |
| Vertigo | 1 (4.8%) | | 1 (4.8%) | |
| Aphthous Stomatitis | 1 (4.8%) | 1 (4.8%) | | |
| Asthenia | 1 (4.8%) | 1 (4.8%) | | |
| Pyrexia | 1 (4.8%) | 1 (4.8%) | | |
| Liver Disorder | 1 (4.8%) | | | 1 (4.8%) |
| Gastroenteritis | 1 (4.8%) | 1 (4.8%) | | |
| Nasopharyngitis | 1 (4.8%) | 1 (4.8%) | | |
| Rhinitis | 1 (4.8%) | 1 (4.8%) | | |
| Neutrophil Count Decreased | 1 (4.8%) | 1 (4.8%) | | |
| Rheumatoid Arthritis | 1 (4.8%) | | | 1 (4.8%) |
| Bronchopneumopathy | 1 (4.8%) | | | 1 (4.8%) |
| Pleural Effusion | 1 (4.8%) | | | 1 (4.8%) |
| Eczema | 1 (4.8%) | 1 (4.8%) | | |
| Onychoclasis | 1 (4.8%) | 1 (4.8%) | | |
| Photosensitivity Reaction | 1 (4.8%) | 1 (4.8%) | | |

Clinical Efficacy of Masitinib

Evaluation of the primary efficacy endpoint ACR and secondary endpoints of ACRn, DAS28 and CRP improvement, are presented in Table 3 according to the ITT LOCF and PP OC analysis groups. Treatment with masitinib significantly improved the severity of active RA as is evident from the week 12 ACR20, ACR50 and ACR70 scores of 15/27 (55.6%), 9/27 (33.3%), and 3/27 (11.1%), respectively in the PP OC group. Correspondingly, the ITT LOCF group scores were 21/39 (53.8%), 10/39 (25.6%), and 3/39 (7.7%). These results are presented as the cumulative number of patients reaching each ACR level, with performance observed to be similar between efficacy analysis groups; the slightly lower response in ITT LOCF was attributable to the fact that imputed data was typically associated to patient withdrawal and therefore, a lower treatment exposure. Considerable improvement was also observed in the ACRn analysis, the PP OC and ITT LOCF analysis groups achieving an improvement of 31.6 and 23.0 units at week 12, respectively. Considering DAS28 scores, the PP OC and ITT LOCF populations exhibited an absolute change of 2.0 and 1.7 units respectively, from a baseline of 6.5 units, representing an improvement in DAS28 classification from “very active RA” to one of “moderate RA”. Regarding the number of patients with a DAS28 score of <2.6 (classified as disease remission), two patients from the ITT LOCF population's MIX subgroup exhibited this improvement but none from the anti-TNFα subgroup. Finally, approximately 50% of patients experienced a significant reduction (>50%) in their CRP levels, signifying a decrease in their inflammation.

The pattern of masitinib efficacy appears independent of previous treatment failure, with approximately 50% of patients achieving the ARC20 and ΔCRP>50% response criteria regardless of previous treatment (Table 3), i.e. masitinib is equally effective in patients for whom previous treatment with anti-TNFα or MTX has been inadequate. Preliminary results from the extension phase are of major interest since they reveal the observed improvement to be consistently maintained over a duration of >84 weeks, demonstrating masitinib's sustainability (Table 4). Regarding the DAS28 extension phase data after 1 year of treatment (60 weeks), an increasing number of patients were achieving DAS28 scores of ≦3.2 or <2.6, signifying inactive RA or an increased likelihood of being in remission. Furthermore, over this time two patients achieved up to 90% improvement (ACR90). Taken together, this suggests that further therapeutic gains could possibly be achieved given longer exposure times.

TABLE 3
Summary of efficacy outcomes at week 12 with subgroup analysis according to previous treatment failure

| | PP OC | | | ITT LOCF | | |
|---|---|---|---|---|---|---|
| | All | Resistance | Resistance | All | Resistance | Resistance |
| Parameter | patients | to anti-TNFα | to MTX | patients | to anti-TNFα | to MTX |
| ACR* | (n = 27) | (n = 14) | (n = 23) | (n = 39) | (n = 19) | (n = 32) |
| ACR20 | 15/27 (55.6%) | 8/14 (57.1%) | 14/23 (60.9%) | 21/39 (53.8%) | 10/19 (52.6%) | 17/32 (53.1%) |
| ACR50 | 9/27 (33.3%) | 4/14 (28.6%) | 9/23 (39.1%) | 10/39 (25.6%) | 4/19 (21.1%) | 9/32 (28.1%) |
| ACR70 | 3/27 (11.1%) | 1/14 (7.1%) | 3/23 (13.0%) | 3/39 (7.7%) | 1/19 (5.3%) | 3/32 (9.4%) |
| ACRn Mean ± SD | 31.6 ± 33 5 | 28.1 ± 32.1 | 36.6 ± 31.6 | 23.0 ± 37.5 | 18.7 ± 36.8 | 24.1 ± 38.8 |
| Median | 42.9 | 44.3 | 46.9 | 25.7 | 20.6 | 32.7 |
| Min-Max | −40.0-87.5 | 40.0-72.2 | −40.0-87.5 | −62.5-87.5 | −62.5-72.2 | −62.5-87.5 |
| CRP | (n = 28) | (n = 14) | (n = 23) | (n = 35) | (n = 17) | (n = 29) |
| Improvement >50% | 14/28 (50.0%) | 7/14 (50.0%) | 12/23 (52.2%) | 19/35 (54.3%) | 9/17 (52.9%) | 16/29 (55.2%) |
| 25% < Improvement ≦50% | 3/28 (10.7%) | 1/14 (7.1%) | 2/23 (8.7%) | 4/35 (11.4%) | 2/17 (11.8%) | 3/29 (10.3%) |
| 0% ≦ Improvement ≦25% | 5/28 (17.9%) | 1/14 (7.1%) | 3/23 (13.0%) | 5/35 (14.3%) | 1/17 (5.9%) | 3/29 (10.3%) |
| Stability | 3/28 (11%) | 3/14 (21%) | 3/23 (13%) | 3/35 (9%) | 3/17 (18%) | 3/29 (10%) |
| Deterioration | 3/28 (11%) | 2/14 (14%) | 3/23 (13%) | 4/35 (11%) | 2/17 (12%) | 4/29 (14%) |
| DAS28 | (n = 24) | (n = 13) | (n = 20) | (n = 34) | (n = 18) | (n = 28) |
| Mean ± SD | 4.6 ± 1.3 | 5.1 ± 1.2 | 4.6 ± 1.4 | 4.8 ± 1.5 | 5.2 ± 1.1 | 4.8 ± 1.5 |
| ΔDAS28 | 2.0 | 1.8 | 2.1 | 1.7 | 1.7 | 1.8 |
| Min-Max | 0.5-7.0 | 3.3-7.0 | 0.5-7.0 | 0.5-7.0 | 3.3-7.0 | 0.5-7.0 |
| DAS28 <2.6 | 1/24 (4.2%) | 0/28 (0%) | 1/20 (5.0%) | 2/34 (5.9%) | 0/18 (0%) | 2/28 (7.1%) |
| DAS28 ≦3.2 | 1/24 (4.2%) | 0/28 (0%) | 1/20 (5.0%) | 2/34 (5.9%) | 0/18 (0%) | 2/28 (7.1%) |

*Primary efficacy outcome. ACR results are presented as the cumulative number of patients reaching each ACR level. ΔDAS28 = the change in DAS28 score from baseline. ITT = intention to treat population, PP = per protocol population, OC = observed cases, LOCF = last observation carried forward.
Remark: Population sizes could vary with respect to an efficacy endpoint due to the fact that for some patients all efficacy data under treatment were missing (no data imputation were possible in this case).

TABLE 4
Efficacy outcomes* from the study's extension phase, week 12 to week 82 (ITT population)

| Parameter | W 12 | W 24 | W 36 | W 48 | W 60 | W 72 | W 84 |
|---|---|---|---|---|---|---|---|
| ACR score (n) | 27 | 7 | 9 | 8 | 8 | 9 | 8 |
| ^{†}ACR20 | 15 (56%) | 6 (86%) | 7 (78%) | 5 (63%) | 6 (75%) | 6 (67%) | 7 (88%) |
| ^{†}ACR50 | 9 (33%) | 2 (27%) | 4 (44%) | 3 (38%) | 6 (75%) | 3 (33%) | 5 (63%) |
| ACR70 | 3 (11%) | 1 (14%) | 2 (22%) | 1 (13%) | 3 (38%) | 2 (22%) | 2 (25%) |
| ACR90 | 0 (0%) | 0 (0%) | 1 (11%) | 0 (0%) | 2 (25%) | 1 (11%) | 1 (13%) |
| ACRn (n) | 27 | 7 | 9 | 8 | 8 | 9 | 8 |
| Mean ± SD | 31.6 ± 33.5 | 36.0 ± 29.0 | 45.9 ± 32.3 | 30.9 ± 36.7 | 58.3 ± 31.4 | 35.6 ± 41.3 | 50.9 ± 38.0 |
| Median | 42.9 | 40.7 | 45.5 | 40.0 | 64.9 | 39.7 | 55.0 |
| Min-Max | −40.0-87.5 | −16.7-73.0 | −3.8-93.3 | −20.0-70.9 | 10.0-93.3 | −27.8-97.4 | −17.6-98.8 |
| CRP (n) | 28 | 7 | 12 | 9 | 7 | 9 | 8 |
| Improvement >50% | 14 (50%) | 5 (71%) | 9 (75%) | 6 (67%) | 3 (43%) | 6 (68%) | 5 (63%) |
| 25% < Improvement ≦50% | 3 (11%) | 0 (0%) | 1 (8%) | 0 (0%) | 3 (43%) | 1 (11%) | 1 (13%) |
| 0% < Improvement ≦25% | 5 (18%) | 1 (14%) | 1 (8%) | 1 (11%) | 1 (14%) | 1 (11%) | 1 (13%) |
| Stable | 3 (11%) | 1 (14%) | 1 (8%) | 0 (0%) | 0 (0%) | 0 (0%) | 0 (0%) |
| Deterioration | 3 (11%) | 0 (0%) | 0 (0%) | 2 (22%) | 0 (0%) | 1 (11%) | 1 (13%) |
| DAS28 (n) | 24 | 4 | 5 | 6 | 7 | 7 | 4 |
| Mean ± SD | 4.6 ± 1.3 | 5.2 ± 1.7 | 4.4 ± 1.9 | 4.7 ± 2.1 | 3.3 ± 1.5 | 3.5 ± 1.5 | 3.1 ± 1.6 |
| Median | 4.4 | 4.9 | 4.1 | 4.4 | 2.6 | 3.0 | 2.5 |
| Min-Max | 0.5-7.0 | 3.6-7.5 | 2.3-7.5 | 2.7-8.7 | 1.7-5.3 | 1.6-6.1 | 1.9-5.5 |
| DAS28 <2.6 | 1 (4.%) | 0 (0%) | 1 (20%) | 0 (0%) | 4 (57%) | 1 (14%) | 2 (50%) |
| DAS28 ≦3.2 | 1 (4%) | 0 (0%) | 1 (20%) | 2 (33%) | 4 (57%) | 4 (57%) | 3 (75%) |

*Results from extension phase are preliminary.
^{†}Primary efficacy outcome. ACR results are presented as the cumulative number of patients reaching each ACR level.

Dose Analysis

Analysis of time to first response according to initial dosage is presented in Table 5. This analysis extends into the extension phase for a total assessment period of approximately 32 weeks. Patients randomized into the 6 mg/kg/day dosing group achieved a response faster than those assigned to the 3 mg/kg/day, (ACR20: median of 29 days against 56 days [p=0.231]; ACR50: 72.5 days against 84 days [p=0.771], respectively); however, these differences were not statistically significant (p<0.05). In cases of an insufficient treatment response, dose adjustment was permitted at weeks 4 and 8; hence, the dose at time of first response was also analyzed. Results reveal that approximately 65% and 73% of those patients achieving ACR20 or ACR50 scores respectively did so at a dosage mg/kg/day. Moreover, this dosage corresponded to the highest response rate (5/15, 33.3%) for the ACR50 threshold. For those patients randomized into the 3 mg/kg/day dosing group, 12/22 (55%) received dose augmentation at weeks 4 or 8 due to insufficient response. Of these, 7/12 patients (58%) experienced an improved response within the initial 12-week phase, while 5/12 patients (42%) were non-responders, having failed to reach the ACR20 threshold.

TABLE 5
Time to first response (days) in ITT population, according to initial dosage

| | Masitinib | Masitinib | Total | |
|---|---|---|---|---|
| | 3 mg/kg/day | 6 mg/kg/day | population | |
| Parameter | (n = 22) | (n = 18) | (n = 40) | p-value |
| ACR20 | | | | |
| Patients | 12/22 (55.0%) | 11/18 (61.0%) | 23/40 (57.5%) | 0.213 |
| Mean ± SD | 51.9 ± 24.5 | 40.3 ± 19.0 | 46.3 ± 22.4 | |
| Median | 56.0 | 29.0 | 35.0 | |
| Min-Max | 28.0-105.0 | 28.0-86.0 | 28.0-105.0 | |
| ACR50 | | | | |
| Patients | 7/22 (32.0%) | 8/18 (44.0%) | 15/40 (37.5%) | 0.771 |
| Mean ± SD | 91.9 ± 59.5 | 86.8 ± 61.1 | 89.1 ± 58.2 | |
| Median | 84.0 | 72.5 | 84.0 | |
| Min-Max | 28.0-217.0 | 28.0-203.0 | 28.0-217.0 | |

interruption. Moreover, because this was the first study of masitinib as treatment in a non-oncologic pathology, the increased incidence of dermatological events typical associated with this therapeutic class was understandably treated with great caution by patients and investigators alike. This may in part explain the relatively high dropout rate of patients. Of those who withdrew from the study because of AEs prior to week 12 (N=13), 9/13 (69%) patients had experienced AEs of a mild or moderate intensity, which could feasibly have been managed without permanent interruption of treatment. In general AEs occurred early during the course of treatment, which is consistent with the known safety profile of tyrosine kinase inhibitors. This trend is clearly evident when comparing safety data from the initial and extension phases, the implication being that although masitinib is not completely free from side effects, the majority of these are over following 12 weeks of treatment with good tolerance experienced thereafter during any long-term treatment regimen. During the initial 12 weeks, the most common AEs were rashes, edema, nausea, and diarrhea. Cutaneous rash may potentially be linked to the action of masitinib on MCs, inducing MC apoptosis with a subsequent release of various mediators, (e.g. histamine, prostaglandins or cytokines), that are responsible for rash. This apoptosis seems to happen only once. The time necessary for the released mediators to reach the reaction site and accumulate to a certain concentration in the skin, might explain why such events typically manifest themselves between the second and third week of treatment. Diarrhea may also be linked to the pharmacological activity of masitinib on MCs in the intestine or through direct action on Cajals cells of the intestine, which also express the c-KIT receptor. Edema, mainly palpebral and face edema, is thought to be linked to the activity of masitinib on the PDGF receptor, a TK receptor involved in the vasculatory pressure of tissues, especially in the periorbital region sensible to low pressure.

Overall, the safety profile of masitinib for long-term treatment would appear favorably, especially when considering that masitinib may exhibit a better safety profile than other tyrosine kinase inhibitors, particularly on cardiotoxicity and genotoxicity (Dubreuil et al., 2009).

The performance of masitinib, with respect to the primary endpoint ACR scores, compares favorably to other biological DMARDs, including rituximab, abatacept, and adalimumab. Moreover, some patients may not have benefited from an optimal masitinib dose due to a lack of dosage increase in case of insufficient response without toxicity (a protocol deviation), and a consequential reduction in efficacy results. Observed clinical improvement was supported by laboratory evidence of reduced inflammation, in the form of a significant and sustainable decrease in the CRP level for approximately half the study population. This result is of importance since in the absence of a control group it serves as proof that the observed improvements are attributable to the treatment. The results from other secondary endpoints, (ACRn and DAS28), provide additional evidence of efficacy, with consistent patterns to the primary endpoint regarding sustainability and independence from previous treatment failure.

Dose response analyses indicate that a dose level of 6 mg/kg/day is the most potent. Considering tolerability, the majority of severe AEs were associated with doses ≧7.5 mg/kg/day. Thus, utilization of ≦6 mg/kg/day would likely reduce the occurrence of severe AEs, in particular those associated with edema.

Conclusions

This study shows that RA patients treated with masitinib, an oral tyrosine kinase inhibitor that acts on MCs, showed positive response in some relevant measures of their condition. Moreover, this positive action was observed in patients with DMARD-refractory active RA, for whom standard treatments including MTX and anti-TNFα were ineffective. This proof-of-concept data supports a confirmatory phase 2b/3 clinical trial to further evaluate the efficacy and safety of masitinib versus placebo in patients suffering from RA. Accordingly, a tyrosine kinase or MC inhibitor such as masitinib is considered to be active in the treatment of human RA, and in particular in the treatment of patients with DMARD-refractory active RA, with an unexpectedly good response.

### Example 2

Masitinib in Combination with Methotrexate in the Treatment of DMARD-Refractory Active Rheumatoid Arthritis

A smaller second study was also conducted to evaluate the safety and efficacy of masitinib in combination with MTX in the treatment of DMARD-refractory active RA.

Study Design and Treatment

A 12-week with possible extension, prospective, multicenter, open label, uncontrolled, 2-parallel group study to compare efficacy and safety of masitinib at 3 and 6 mg/kg/day in combination with MTX, in treatment of patients with active RA with inadequate response to: (1) MTX; (2) any DMARD including anti-TNFα if patients previously failed MTX; or (3) MTX in combination with any DMARD including anti-TNFα.

Summary of Results

After 12 weeks of treatment, three out of 8 patients (37.5% of study population) were evaluable for ACR.
One patient achieved ACR20 at week 8 through 12 without further improvement.
One patient reached ACR20 at week 12.
One patient achieved ACR70 at week 4 that was maintained up to week 12, This patient had failed anti-TNFα treatment and received the masitinib dosage of 6 mg/kg/day.

All these patients experienced a decrease in their CRP level at week 12; two patients showed a CRP score improvement of between 25 and 50%, and one patient (treated with 6 mg/kg/day of masitinib) exhibited good improvement greater than 50%.

The analysis of individual parameters at week 12 in total ITT population (n=8) revealed that:
There was a good improvement of general patient health (+92.2%);
HAQ assessment improved by 29.5%;
Hamilton Score improved by 75.7%;
Pain assessment decreased (mean of −72.2%);
Asthenia assessment decreased (mean of −19.6%);
Mean FIS Physical and Social score slightly decreased;
FIS cognitive score decreased by 145%.

Conclusions

This study shows that RA patients treated with masitinib in combination with MTX, achieved positive response in some relevant measures of their condition. Moreover, this positive action was observed in patients with DMARD-refractory active RA. This proof-of-concept data supports a confirmatory phase 2b/3 clinical trial to further evaluate the efficacy and safety of masitinib versus placebo in patients suffering from RA.

Accordingly, a tyrosine kinase or MC inhibitor such as masitinib is considered to be active in the treatment of human RA, including patients with DMARD-refractory active RA, especially when administered in combination with MTX, which provides unexpectedly good results.

### REFERENCES

ACR Clinical Symposia. Rheumatoid Arthritis (RA): Ideal Management From Start To Finish—Integrating New ACR/European League Against Rheumatism (EULAR) Criteria. Program and abstracts of the American College of Rheumatology 2009 Annual Meeting; Oct. 17-21, 2009; Philadelphia, Pa.
Arnett F C, Edworthy S M, Bloch D A, McShane D J, Fries J F, Cooper N S, Healey L A, Kaplan S R, Liang M H, Luthra H S, Medsger T A Jr, Mitchell D M, Neustadt D H, Pinals R S, Schaller J G, Sharp J T, Wilder R L, Hunder G G: The American Rheumatism Association 1987 revised criteria for the classification of rheumatoid arthritis. Arthritis Rheum 1988, 31:315-324.
Dubreuil P, Letard S, Ciufolini M, Gros L, Humbert M, Castéran N, Borge L, Hajem B, Lermet A, Sippl W, Voisset E, Arock M, Auclair C, Leventhal P S, Mansfield C D, Moussy A, Hermine O (2009) Masitinib (AB1010), a potent and selective tyrosine kinase inhibitor targeting KIT. PLoSONE 4(9): e7258. doi:10.1371/journal.pone.0007258.
Felson D T, Anderson J J, Boers M, Bombardier C, Chemoff M, Fried B, et al, and the Committee on Outcome Measures in Rheumatoid Arthritis Clinical Trials. The American College of Rheumatology preliminary core set of disease activity measures for rheumatoid arthritis clinical trials. Arthritis Rheum 1993; 36:729-40.
Gilfillan A M, Tkaczyk C. Integrated signalling pathways for mast-cell activation. Nature Review Immunology 2006; 6: 218-230.
Hochberg M C, Chang R W, Dwosh I, Lindsey S, Pincus T, Wolfe F. The American College of Rheumatology 1991 revised criteria for the classification of global functional status in rheumatoid arthritis. Arthritis Rheum 1992; 35:498-502.
Lee D M, Friend D S, Gurish M F, Benoist C, Mathis D, Brenner M B: Mast cells: a cellular link between autoantibodies and inflammatory arthritis. Science 2002, 297:1689-1692.
Nigrovic P A, Binstadt B A, Monach P A, Johnsen A, Gurish M, Iwakura Y, Benoist C, Mathis D, Lee D M: Mast cells contribute to initiation of autoantibody-mediated arthritis via IL-1. Proc Natl Acad Sci USA 2007, 104:2325-2330.
Reber L, DaSilva C A, Frossard N: Stem cell factor and its receptor c-Kit as targets for inflammatory diseases. Eur J Pharmacol 2006, 533:327-40.
Tebib, X Mariette, P Bourgeois, R M Flipo, P Gaudin, X Le Loet, P Gineste, L Guy, CD Mansfield, A Moussy, P Dubreuil, O Hermine, J Sibilia, “Masitinib in the treatment of active rheumatoid arthritis: results of a multicentre, open-label, dose-ranging, phase 2a study”, Arthritis Res Ther, 11:R95 doi:10.1186/ar2740, 2009
van der Heijde D M, van 't H of M, van Riel P L, van de Putte L B. Development of a disease activity score based on judgment in clinical practice by rheumatologists. J Rheumatol 1993 March; 20(3):579-81

1-38. (canceled)

39. A method of treatment of rheumatoid arthritis in human patients, comprising administering to said patients a tyrosine kinase inhibitor or a mast cell inhibitorat a starting dose of 3.0 to 7.5±1.5 mg/kg/day.

40. The method according to claim 39, wherein said patients are diagnosed as having ‘definite’ or ‘probable’ rheumatoid arthritis according to the ACR/EULAR classification systems and an ACR global functional status of class I to III.

41. The method according to claim 39, wherein said method is for treating DMARD-resistant rheumatoid arthritis.

42. The method according to claim 39, comprising administering to the patients the tyrosine kinase inhibitor or mast cell inhibitorat a starting dose of 4.5 to 7.5±1.5 mg/kg/day.

43. The method according to claim 39, wherein administration of said tyrosine kinase inhibitor or mast cell inhibitor is dose escalated by increments of 1.5 mg/kg/day to reach a maximum of 9.0 mg/kg/day.

44. The method according to claim 39, wherein said tyrosine kinase inhibitor or mast cell inhibitor is administered orally.

45. The method according to claim 39, wherein said tyrosine kinase inhibitor or mast cell inhibitor is administered twice a day.

46. The method according to claim 39, comprising administering an effective amount of said tyrosine kinase inhibitor or mast cell inhibitor over more than 3 months.

47. The method according to claim 46, comprising administering an effective amount of said tyrosine kinase inhibitor or mast cell inhibitor over more than 12 months.

48. The method according to claim 39, comprising administering a pharmaceutical composition comprising a dose of at least 50 mg and less than 150 mg of said tyrosine kinase inhibitor or mast cell inhibitor.

49. The method according to claim 48, wherein the dose is 100 mg.

50. The method according to claim 39 comprising administering a pharmaceutical composition comprising a dose of at least 150 mg and less than 400 mg of said tyrosine kinase inhibitor or mast cell inhibitor.

51. The method according to claim 50, wherein the dose is 200 mg.

52. The method according to claim 39, wherein said tyrosine kinase inhibitor or mast cell inhibitor is administered in combination with at least one antirheumatic drug.

53. The method according to claim 52, wherein the antirheumatic drug is selected from the group consisting of: non-biological DMARDs; biological DMARDs; corticosteroids; non-steroidal anti-inflammatory drugs; or anti-inflammatory steroidal drugs.

54. The method according to claim 53, wherein the antirheumatic drug is methotrexate.

55. The method according to claim 53, wherein the antirheumatic drug is a TNFα blocker.

56. The method according to claim 53, wherein the antirheumatic drug is prednisone.

57. The method according to claim 52, wherein the tyrosine kinase inhibitor or mast cell inhibitor and the antirheumatic drug(s) are administered separately, simultaneously or sequentially in time.

58. The method according to claim 39, wherein said tyrosine kinase inhibitor or a mast cell inhibitor is masitinib or a pharmaceutically acceptable salt thereof.

59. The method according to claim 58, wherein masitinib is masitinib mesylate.
